(19) United States
(12) Patent Application Publication
(10) Pub. No.: US 2026/0031219 A1
(43) Pub. Date: Jan. 29, 2026

(54) PREDICTING PATIENT STAYS FOR ASSET MANAGEMENT

(71) Applicant: Hill-Rom Services, Inc., Batesville, IN (US)

(72) Inventors: Alexis Balingcongan, Wesley Chapel, FL (US); Sarah Blanck, Highland Park, IL (US); Anandi Mahadevan, Murrysville, PA (US)

(21) Appl. No.: 19/277,708

(22) Filed: Jul. 23, 2025

Related U.S. Application Data
(60) Provisional application No. 63/675,876, filed on Jul. 26, 2024.

(51) Int. Cl. G16H 40/20; G16H 50/30; G16H 70/20; G16H 80/00

(52) U.S. Cl. CPC G16H 40/20; G16H 50/30; G16H 70/20; G16H 80/00

(57) ABSTRACT

An example computer system for predicting a length of stay of a patient for asset management can include: one or more processors; and non-transitory computer-readable storage media encoding instructions which, when executed by the one or more processors, causes the computer system to: train a length of stay model to estimate the length of stay of the patient; train an asset model for managing assets of a facility; and pair the length of stay model and the asset model to optimize management of the assets for the patient.

### BACKGROUND

Care facilities (like hospitals) can have barriers to maintaining room availability in the hospital setting. One of those barriers is having the right assets in place when the need arises for them. If the assets are not available, room availability can become stalled, creating unnecessary backups and delays to treatment and eventually to elongated length of stays at the hospital.

### SUMMARY

Examples provided herein are directed to predicting patients stay for asset management.

According to one aspect, an example computer system for predicting a length of stay of a patient for asset management can include: one or more processors; and non-transitory computer-readable storage media encoding instructions which, when executed by the one or more processors, causes the computer system to: train a length of stay model to estimate the length of stay of the patient; train an asset model for managing assets of a facility; and associate the length of stay model and the asset model to optimize management of the assets for the patient.

According to another aspect, an example method for predicting a length of stay of a patient for asset management can include: training a length of stay model to estimate the length of stay of the patient; training an asset model for managing assets of a facility; and associating the length of stay model and the asset model to optimize management of the assets for the patient.

The details of one or more techniques are set forth in the accompanying drawings and the description below. Other features, objects, and advantages of these techniques will be apparent from the description, drawings, and claims.

### DESCRIPTION OF THE DRAWINGS

FIG. 1 shows an example system for predicting patient stays for asset management for a care facility.

FIG. 2 shows example logical components of a server device of the system of FIG. 1.

FIG. 3 shows an example method executed by the server device of FIG. 2.

FIG. 4 shows example physical components of the server device of FIG. 2.

### DETAILED DESCRIPTION

This disclosure relates to predicting patient stays for asset management.

In the examples provided herein, artificial intelligence is used to predict a length of stay/date of discharge for a patient using claims data and other patient data. This allows a prediction of the potential path of a patient from admission to a care facility (e.g., through critical care, operating room, emergency department, etc.) and all the way to discharge. This information can be paired or otherwise associated with the facilities asset tracking system to ensure assets and equipment are available as needed for the patient.

The concept can pair what is known about the patient and the care facility's assets using modeling so that they match at the right stages of the patient's journey. This allows the facility to have equipment in the right location at the right time for timely transfers and patient care. Such information can impact the availability of beds, equipment, etc. for the patients. This availability is impacted by the length each patient is in the hospital, along with the difference phases of care for each patient.

There can be various advantages associated with the technologies described herein. For instance, the GenAI and modeling can be trained to provide a more efficient system for care. This results in better use of assets and more timely care provided to the patients. Further, the modeling can be trained to become better over time and with more inputs, thereby enhancing the effects of asset management and resulting in the practical application of the technology.

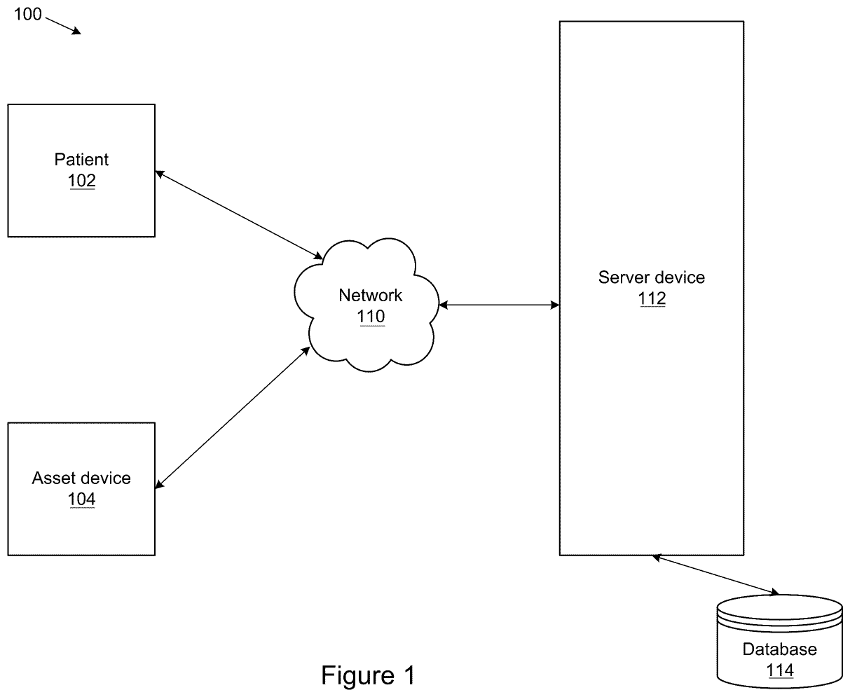

FIG. 1 schematically shows aspects of one example system 100 programmed to predict patient stays for asset management. In this example, the system 100 can be a computing environment that includes a plurality of client and server devices. In this instance, the system 100 includes a patient 102, an asset device 104, a server device 112, and a database 114. The asset device 104 can communicate with the server device 112 through a network 110 to accomplish the functionality described herein.

Each of the devices may be implemented as one or more computing devices with at least one processor and memory. Example computing devices include a mobile computer, a desktop computer, a server computer, or other computing device or devices such as a server farm or cloud computing used to generate or receive data.

In some non-limiting examples, the server device 112 is owned by a care facility, such as a hospital. The asset device 104 can be programmed to communicate with the server device 112 to allow the server device 112 to predict patient stay to manage the asset device 104 accordingly. In this example, the server device 112 implements SmartCare Remote Management from Hill-Rom Holdings, Inc., which allows the server device 112 to manage the asset device 104, as described further below. Many other configurations are possible.

The example patient 102 is a patient of the system 100. In some instances, the patient 102 is an in-patient of the hospital which operates the system 100. Information about the patient 102 is captured by the system 100, such as using an Electronic Medical Record (EMR) system. While only a single patient is shown, a typical hospital can treat hundreds or thousands of patients.

The example asset device 104 is programmed to deliver care to the patient 102. The asset device 104 can take a wide variety of forms, such as a bed for the patient (e.g., a Progressa+ Bed from Hill-Rom Holdings, Inc.), a patient monitoring device (e.g., a Connex Vital Signs Monitor from Hill-Rom Holdings, Inc.), and/or a diagnostic device (e.g., a Retina Vue 700 Imager from Hill-Rom Holdings, Inc.). These are but a few of the hundreds or thousands of assets that can be used to deliver care to the patient 102.

The example server device 112 is programmed to manage the care provided to the patient 102. This can include, without limitation, predicting a length of stay for the patient 102 within the system 100. The server device 112 can also be programmed to predict the assets needed to provide care to the patient 102. Further, as provided below, the server device 112 is programmed to associate the predicted length of stay and asset management for the patient 102 to optimize the care given to the patient 102 and other patients in the system 100.

The example database 114 is programmed to store data for the system 100. This data can include, for instance, the EMR system. The database 114 can also store the models used to predict the length of stay and assets needed for each of the patients in the system 100, including the patient 102.

The network 110 provides a wired and/or wireless connection between the asset device 104 and the server device 112. In some examples, the network 110 can be a local area network, a wide area network, the Internet, or a mixture thereof. Many different communication protocols can be used. Although only two devices are shown, the system 100 can accommodate hundreds, thousands, or more of computing devices. For instance, there may be thousands of assets associated with the system 100 for patient care.

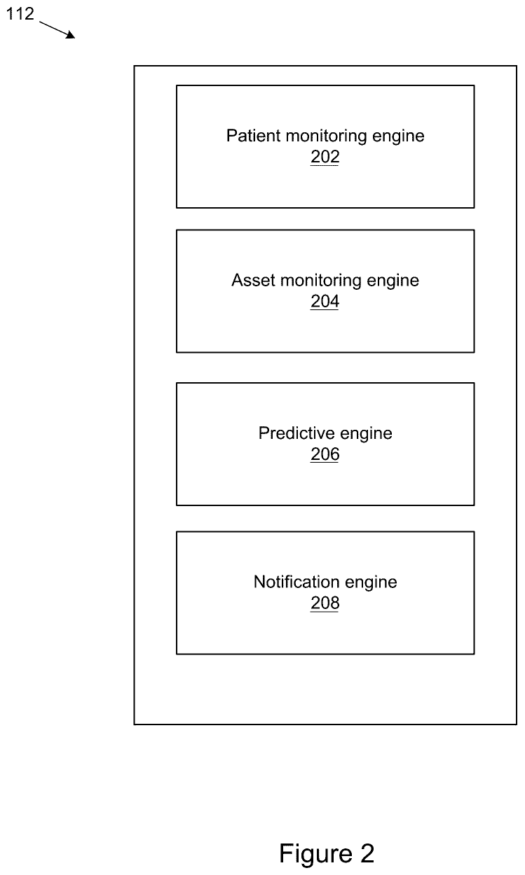

Referring now to FIG. 2, additional details of the server device 112 are shown. In this example, the server device 112 has various logical engines that assist in predicting patient stays for asset management. The server device 112 can, in this instance, include a patient monitoring engine 202, an asset monitoring engine 204, a predictive engine 206, and a notification engine 208. In other examples, more or fewer engines providing different functionality can be used.

The patient monitoring engine 202 is programmed to monitor various aspects of a patient's stay within the hospital to make predictions described below. In some examples, the patient monitoring engine 202 defines a model that is used to predict the length of stay for the patient 102.

For instance, the patient monitoring engine 202 is programmed to train a length of stay model using benchmark hospital national averages for particular procedures for patients. In this example, the patient monitoring engine 202 uses International Classification of Diseases, Tenth Revision (ICD-10) diagnosis codes for the training. The patient monitoring engine 202 can also consider other information when training the length of stay model, such as information specific to the hospital, such as success rates, infection rates, etc.

The asset monitoring engine 204 is programmed to monitor various aspects of the assets within the hospital to make predictions described below. In some examples, the patient monitoring engine 202 defines a model that is used to predict the need for one or more assets to treat the patient 102.

For instance, the asset monitoring engine 204 is programmed to train an asset model that is trained using the various assets provided by the hospital within the system 100, such as the asset device 104. The asset model can be trained to understand various aspects associated with the asset device 104, such as: the use cases for the asset device 104; any dependencies on the use; and/or where the asset device 104 is located on a map (e.g., using the ReadyConnect wireless connection system from Hill-Rom Holdings, Inc.), etc.

The predictive engine 206 is programmed to use GenAI to associate the length of stay model from the patient monitoring engine 202 and the asset model from the asset monitoring engine 204 to make predictions about patients and manage the assets for the system 100. For instance, the predictive engine 206 can generate one or more GenAI queries to estimate the length of stay for the patient 102, and thereupon optimize the asset device 104 needed to provide care for the patient 102 during the stay.

For instance, assume the patient 102 is admitted to the hospital for removal of her appendix. The predictive engine 206 would use the appropriate ICD-10 code associated with this procedure to estimate the length of stay for the patient 102. The predictive engine 206 would then manage the assets needed to provide the care for the patient 102 over that stay. Such assets could include the bed for the patient 102, the patient monitor to monitor the vital signs of the patient 102, and a drug pump to deliver drugs to the patient 102. By using the prediction of the length of stay for the patient 102 from the length of stay model, the predictive engine 206 can pair that with the asset model to assure that these assets are available at the necessary time and for the necessary durations during the stay of the patient 102.

In some examples, a caregiver for the patient 102 can provide input into the length of stay and/or assets needed for the care. For instance, the caregiver can be presented with a checklist for all assets needed for the patient 102, and the input from the caregiver can be further used by the predictive engine 206 to estimate the length of stay and manage the assets, like the asset device 104.

Further, additional input about the patient 102 can be fed back into the predictive engine 206 to update the estimates based upon a patient risk assessment. For instance, the various early warning scores, such as a patient risk surveillance score, can be fed into the predictive engine 206 to determine possible deterioration or improvement by the patient 102. This can impact the estimated length of stay and possible assets needed by the patient 102.

The notification engine 208 is programmed to communicate the estimates about the length of stay and assets needed.

For instance, the notification engine 208 can communicate with family members, providers, and other individuals associated with the patient 102 as milestones associated with the care of the patient 102 are reached. Such information can be sent through the Voalte Platform from Hill-Rom Holdings, Inc., which allows for communication between patients, caregivers, and family members. For instance, the notification engine 208 can provide the estimated length of stay for the patient 102 to family members so the family members know when to expect the patient 102 to be discharged.

The notification engine 208 can also manage the assets needed for the care of the patient 102. For instance, when the asset device 104 is needed for the patient 102, the notification engine 208 can send a notification to the caregiver or otherwise coordinate the location of the asset device 104. Further, the notification engine 208 can predict when the asset device 104 will be available for other patients after the patient 102 has received the necessary care.

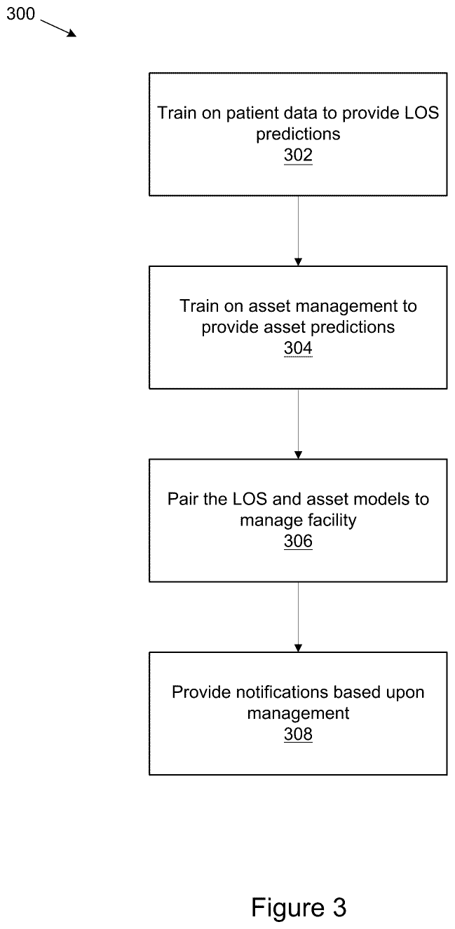

FIG. 3 illustrate an example method 300 for predicting patient stay for asset management as executed by the server device 112 of the system 100.

In operation 302 of the method 300, the length of stay model is trained on patient data to provide length of stay determinations. For instance, as described above, the model can be trained on national data to estimate length of stays.

In operation 304, the asset model is trained on asset data to provide management of the assets in the system 100. For instance, as described above, the model can be trained on asset data that is used to determine information like availability, location, length of use, etc.

Next, at operation 306, the length of stay model and the asset model are associated to provide an overall management of the patients and assets within the hospital. As noted previously, this information can be used to manage the assets within the hospital to provide optimized patient care.

Finally, at operation 308, notifications are provided based upon that management. For instance, the patient 102 and family members can be notified of the estimate of the length of stay. Further, the assets necessary for patient care can be managed through notifications based upon need, use, and availability.

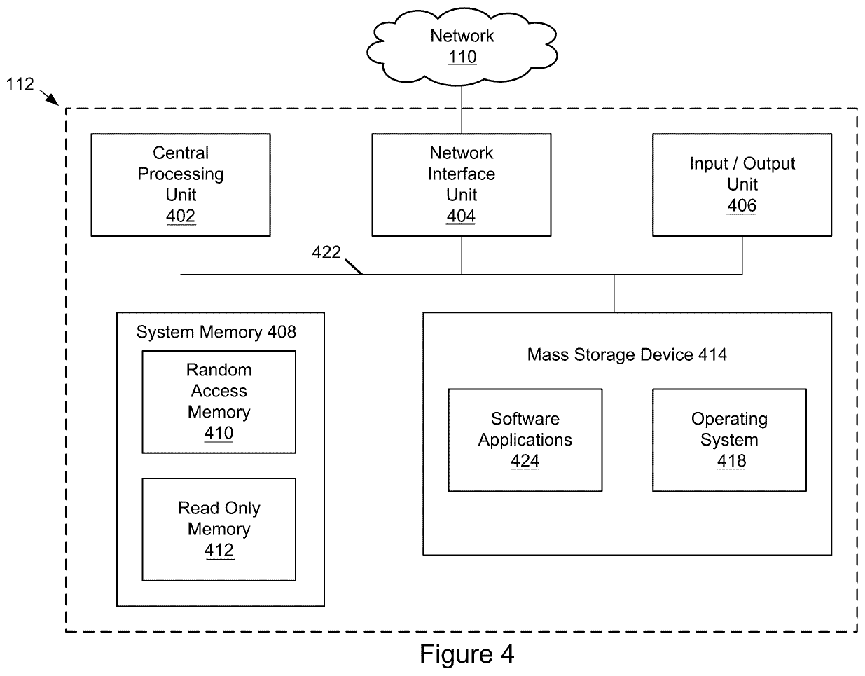

As illustrated in the embodiment of FIG. 4, the example server device 112, which provides the functionality described herein, can include at least one central processing unit (“CPU”) 402, a system memory 408, and a system bus 422 that couples the system memory 408 to the CPU 402. The system memory 408 includes a random access memory (“RAM”) 410 and a read-only memory (“ROM”) 412. A basic input/output system containing the basic routines that help transfer information between elements within the server device 112, such as during startup, is stored in the ROM 412. The server device 112 further includes a mass storage device 414. The mass storage device 414 can store software instructions and data. A central processing unit, system memory, and mass storage device similar to that shown can also be included in the other computing devices disclosed herein.

The mass storage device 414 is connected to the CPU 402 through a mass storage controller (not shown) connected to the system bus 422. The mass storage device 414 and its associated computer-readable data storage media provide non-volatile, non-transitory storage for the server device 112. Although the description of computer-readable data storage media contained herein refers to a mass storage device, such as a hard disk or solid-state disk, it should be appreciated by those skilled in the art that computer-readable data storage media can be any available non-transitory, physical device, or article of manufacture from which the central display station can read data and/or instructions.

Computer-readable data storage media include volatile and non-volatile, removable, and non-removable media implemented in any method or technology for storage of information such as computer-readable software instructions, data structures, program modules, or other data. Example types of computer-readable data storage media include, but are not limited to, RAM, ROM, EPROM, EEPROM, flash memory or other solid-state memory technology, CD-ROMs, digital versatile discs (“DVDs”), other optical storage media, magnetic cassettes, magnetic tape, magnetic disk storage or other magnetic storage devices, or any other medium which can be used to store the desired information and which can be accessed by the server device 112.

According to various embodiments of the invention, the server device 112 may operate in a networked environment using logical connections to remote network devices through network 110, such as a wireless network, the Internet, or another type of network. The server device 112 may connect to network 110 through a network interface unit 404 connected to the system bus 422. It should be appreciated that the network interface unit 404 may also be utilized to connect to other types of networks and remote computing systems. The server device 112 also includes an input/output controller 406 for receiving and processing input from a number of other devices, including a touch user interface display screen or another type of input device. Similarly, the input/output controller 406 may provide output to a touch user interface display screen or other output devices.

As mentioned briefly above, the mass storage device 414 and the RAM 410 of the server device 112 can store software instructions and data. The software instructions include an operating system 418 suitable for controlling the operation of the server device 112. The mass storage device 414 and/or the RAM 410 also store software instructions and applications 424, that when executed by the CPU 402, cause the server device 112 to provide the functionality of the server device 112 discussed in this document.

Although various embodiments are described herein, those of ordinary skill in the art will understand that many modifications may be made thereto within the scope of the present disclosure. Accordingly, it is not intended that the scope of the disclosure in any way be limited by the examples provided.

What is claimed is:

1. A computer system for predicting a length of stay of a patient for asset management, comprising:
one or more processors; and
non-transitory computer-readable storage media encoding instructions which, when executed by the one or more processors, causes the computer system to:
train a length of stay model to estimate the length of stay of the patient;
train an asset model for managing assets of a facility; and
associate the length of stay model and the asset model to optimize management of the assets for the patient.

2. The computer system of claim 1, wherein the length of stay model is trained using benchmark current hospital national averages for particular procedures for patients.

3. The computer system of claim 2, wherein the particular procedures are identified using an International Classification of Diseases.

4. The computer system of claim 1, comprising further instructions which, when executed by the one or more processors, causes the computer system to modify the length of stay of the patient based upon a patient risk assessment.

5. The computer system of claim 1, wherein the asset model is configured to include an availability and a location for the assets.

6. The computer system of claim 1, comprising further instructions which, when executed by the one or more processors, causes the computer system to modify the asset model based upon input from a caregiver.

7. The computer system of claim 1, comprising further instructions which, when executed by the one or more processors, causes the computer system to notify the patient of a specific length of stay for the patient based upon the length of stay model.

8. The computer system of claim 7, comprising further instructions which, when executed by the one or more processors, causes the computer system to notify family of the patient of the specific length of stay for the patient based upon the length of stay model.

9. The computer system of claim 1, comprising further instructions which, when executed by the one or more processors, causes the computer system to notify a caregiver of assets needed for the patient.

10. The computer system of claim 1, comprising further instructions which, when executed by the one or more processors, causes the computer system to use generative artificial intelligence to associate the length of stay model and the asset model.

11. A method for predicting a length of stay of a patient for asset management, comprising:
training a length of stay model to estimate the length of stay of the patient;
training an asset model for managing assets of a facility; and
associating the length of stay model and the asset model to optimize management of the assets for the patient.

12. The method of claim 11, wherein the length of stay model is trained using benchmark current hospital national averages for particular procedures for patients.

13. The method of claim 12, wherein the particular procedures are identified using an International Classification of Diseases.

14. The method of claim 11, further comprising modifying the length of stay of the patient based upon a patient risk assessment.

15. The method of claim 11, wherein the asset model is configured to include an availability and a location for the assets.

16. The method of claim 11, further comprising modifying the asset model based upon input from a caregiver.

17. The method of claim 11, further comprising notifying the patient of a specific length of stay for the patient based upon the length of stay model.

18. The method of claim 17, further comprising notifying family of the patient of the specific length of stay for the patient based upon the length of stay model.

19. The method of claim 11, notifying a caregiver of assets needed for the patient.

20. The method of claim 11, further comprising using generative artificial intelligence to associate the length of stay model and the asset model.
